(19) United States
(12) Patent Application Publication
(10) Pub. No.: US 2024/0407402 A1
(43) Pub. Date: Dec. 12, 2024

(54) SYSTEM AND METHOD FOR HEATING AND/OR COOLING, IN PARTICULAR PASTEURIZING, FILLED AND CLOSED CONTAINERS

(71) Applicant: Krones AG, Neutraubling (DE)

(72) Inventors: Stefan KAATZ, Fleckeby (DE); Jesse SCHMUCK, Flensburg (DE)

(21) Appl. No.: 18/734,725

(22) Filed: Jun. 5, 2024

(30) Foreign Application Priority Data
Jun. 6, 2023 (DE) 10 2023 114 816.8

(51) Int. Cl. A23L 3/02; A23L 3/00

(52) U.S. Cl. CPC A23L 3/022; A23L 3/001; A23L 3/003

(57) ABSTRACT

The disclosure relates to a system for heating and/or cooling, in particular pasteurizing, closed containers filled with an in particular liquid filling material. The system comprises a transport device designed to transport the containers in a transport direction, a device designed to heat and/or cool the containers according to a temperature profile along the transport direction, and a control device designed to control a transport speed of the transport device and the temperature profile. The control device is designed to control the transport speed during the time change between a stop and a nominal transport speed and to control the temperature profile as a function of a predicted system parameter. The disclosure also relates to a method for heating and/or cooling the closed containers filled with an in particular liquid filling material by means of such a system.

### CROSS REFERENCE TO RELATED APPLICATION

The present application claims priority to German Patent Application No. 10 2023 114 816.8 filed on Jun. 6, 2023. The entire contents of the above-listed application are hereby incorporated by reference for all purposes.

### TECHNICAL FIELD

The disclosure relates to a system and to a method for heating and/or cooling, in particular pasteurizing, closed containers filled with an in particular liquid filling material.

### BACKGROUND

Devices for heating and/or cooling filled and closed containers are regularly used in automated systems in the consumer goods and/or food industry, for example in the form of a pasteurizer.

### SUMMARY

When the pasteurizer is integrated into an automated system comprising a plurality of successive machines, there is a need to adjust the system parameters of the individual machines, in particular the transport speeds of the individual machines, in order to ensure a smooth overall process.

EP 2 833 742 B1 discloses a device in which a transport speed of a transport element within a pasteurizer can be continuously controlled or regulated. When the transport speed is increased, the number of zones used as preheating zones is reduced while the treatment temperature in these zones is simultaneously increased, and the number of zones used as pasteurization zones is increased. When the transport speed is reduced, the number of zones used as preheating zones is increased, with partial reduction of the treatment temperature, and the number of zones acting as pasteurization zones is reduced.

Alternatively, an approach is known from the prior art in which, in the event of a limited availability of products at the infeed, the pasteurizer stops and resumes at total capacity again as soon as sufficient products are present. Accordingly, the pasteurizer stops in the event of an accumulation at the outfeed and resumes at total capacity when the accumulation is cleared.

In this approach, the process control must assume that total transport capacity is always provided at each start. To ensure adequate product treatment, the temperature of the treatment medium is increased accordingly. However, if there is a temporary standstill of the pasteurizer due to an accumulation and/or limited availability of products at the infeed and/or outfeed, this can result in overpasteurization of the products.

In view of this, the object of the disclosure consists in reducing the energy consumption in the heat treatment of filled and closed containers while maintaining good pasteurization results.

This object is achieved by a system as described herein.

For example, the disclosure provides a system for heating and/or cooling, in particular pasteurizing, containers filled with an in particular liquid filling material and closed. The system comprises a transport device designed to transport the containers in a transport direction, a device designed to heat and/or cool the containers according to a temperature profile along the transport direction, and a control device designed to control a transport speed of the transport device and the temperature profile. The control device is designed to control the transport speed during the time change between a stop and a nominal transport speed and to control the temperature profile as a function of a predicted system parameter.

The system can in particular be a tunnel pasteurizer.

Due to the fact that the transport speed changes between a stop and a nominal transport speed, an adjustment of the temperature profile would result in a rather fast change of the temperatures in the system due only to the current transport speed. However, because the temperatures in the system are rather sluggish compared to the time in which the transport speed changes (even several times), it seems to be better to control the temperature profile based on a predicted system parameter than based on (only) a current system parameter.

The change between a stop and a nominal transport speed takes place, for example, in such a way that only these values are predefined by the controller for the transport speed, and no intermediate values, such as a transport speed of 30% or 50% or 70% of the nominal transport speed.

For a fast change between a stop and the nominal transport speed (compared to the sluggish temperature control system of the system), for example due to utilization of the system at less than its nominal capacity, controlling the temperature profile with a predicted system parameter is better than controlling it based on the comparatively fast changing actual transport speed.

Brief heating to temperatures of 60 to 90° C. can be understood as pasteurization.

In some cases, the liquid filling material is food, In particular juice or milk.

Containers can mean food packaging such as bottles, cans or canisters. The containers can be closed by means of lids and/or closures and/or films.

The transport device can be or comprise a conveyor belt. The transport direction is the direction in which the transport device moves.

The device designed to heat and/or cool the containers is provided, for example, to act on the containers with a liquid treatment medium, in particular water, at a controllable and therefore adjustable temperature. The desired temperature of the treatment medium is determined by the temperature profile.

The control device can generate control signals based on data and in particular transmit the control signals to regulating devices and/or components.

The system parameter can comprise, for example, the transport speed, a number of containers entering in a unit of time, a number of containers that can be output in a unit of time, the length of the full part of an accumulation section, the number of (accumulated) containers in an accumulation section, an accommodation and/or output capacity, and/or a system utilization in percent, in each case of the system.

By controlling the temperature profile as a function of the predicted system parameter, which can be an expression of a throughput of the system of containers per unit of time in the future, an expected amount of energy required for heat treatment of the containers and, accordingly, the temperature profile can be deduced. The underlying object of reducing costs and energy consumption, as well as the negative impact of overpasteurization, is thereby achieved in a simple manner.

Here and below, the accommodation capacity can mean how many containers can be accommodated. Accordingly, here and below, the output capacity can be understood to mean how many containers can be output. The accommodation and/or output capacity can comprise an absolute variable or a relative variable, in particular with respect to the total accumulation capacity of an accumulation section.

Here and in the following, the system utilization is a proportion of an actual throughput of containers per unit of time at a maximum throughput of containers per unit of time. The maximum throughput can be a nominal throughput.

The system parameter can be an average transport speed of the system.

The control device can be designed to control the transport speed as a function of a number of containers in an infeed and/or outfeed of the system in the past and/or present, in particular the time curves thereof, and/or an available capacity for accommodating containers in the/an infeed and/or outfeed of the system in the past and/or present, in particular the time curves thereof.

By controlling the transport speed in this way, an accumulation or defect at the infeed and/or outfeed and thus a critical operational state and/or an operational state with increased energy consumption is avoided.

The system can comprise a detection device in the region of the/an infeed and/or outfeed of the system for detecting containers in the region of the infeed and/or outfeed of the system. The detection device can comprise an accumulation switch and/or an image capturing device, wherein the control device can in particular be designed to control the transport speed based on data of the detection device.

By means of the detection device in the region of the infeed and/or outfeed of the system, data about the system is collected in a simple manner, which data has a direct influence on the operational state of the system. By means of the detection device, it is possible to detect the containers in the region of the infeed and/or outfeed of the system and to control the transport speed as a function of the actual presence of containers in the region of the infeed and/or outfeed, in particular in the event of defects and/or accumulations in the infeed and/or outfeed. Critical operational states and/or operational states with increased energy consumption are thereby avoided in a simple manner.

In some cases, the accumulation switch comprises a movable bracket and a sensor, wherein the bracket is moved as the accumulation pressure increases and the changed position of the bracket is detected by the sensor.

The image capturing device can be a camera, for example.

Data from the detection device can be understood to mean a signal from the sensor of the accumulation switch and/or an image from the image capturing device.

The control device can be designed to predict the system parameter based on a transport speed, a number of containers entering in a unit of time, a number of containers that can be output in a unit of time, the/a length of the full part of an accumulation section, a number of (accumulated) containers in an accumulation section, an accommodation and/or output capacity, and/or a system utilization in percent, in each case of the system, and/or of one or more further systems upstream and/or downstream of the system in the past and/or present.

The system parameter of the system can be reliably predicted due to the dependence of the predicted system parameter of the system on one or more system parameters of the system and/or one or more further systems upstream and/or downstream of the system in the past and/or present. An advantage of this type of prediction is that it requires no data other than known, inherent system parameters.

The device can have a plurality of temperature zones along the transport direction of the containers. The temperature profile can indicate controllable temperatures of the temperature zones. Controlling the temperature profile can comprise controlling the temperatures of the temperature zones, wherein each temperature zone can have a uniform temperature for itself.

The pasteurization of the containers is carried out as needed as a result of the presence of a plurality of temperature zones of the device.

In the system, the temperatures can increase or remain constant from temperature zone to temperature zone in a plurality of temperature zones referred to as heating zones, which can follow one another in the transport direction. The heating zones are used to heat the filling material of the containers. In addition, the temperature can correspond to a maximum temperature (of the temperature profile) in at least one or more temperature zones referred to as pasteurization zones following the heating zones in the transport direction. Pasteurization of the filling material takes place here. In addition, the temperatures can decrease or remain constant from temperature zone to temperature zone in a plurality of temperature zones referred to as cooling zones following the pasteurization zones in the transport direction. The cooling zones are used to cool the filling material of the containers, thereby ending the pasteurization process in a container.

Classification of the temperature zones by function improves control of the heat treatment of the containers.

In the plurality of heating zones, the temperatures can increase stepwise and/or gradually from temperature zone to temperature zone. The temperatures of the heating zones can be lower than the maximum temperature (of the temperature profile).

In the plurality of cooling zones, the temperatures can decrease stepwise and/or gradually from temperature zone to temperature zone. The temperatures of the cooling zones can be lower than the maximum temperature.

The control device can be designed to increase a number of pasteurization zones. This occurs if a) a predicted transport speed, a predicted number of containers entering in a unit of time, a predicted output capacity, and/or a predicted system utilization in percent, in each case of the system, increases, and/or b) a predicted number of containers that can be output in a unit of time, and/or a predicted accommodation capacity, in each case of the system, decreases.

Alternatively or additionally, the control device can be designed to reduce a/the number of pasteurization zones if a) a predicted transport speed, a predicted number of containers entering in a unit of time, a predicted output capacity, and/or a predicted system utilization in percent, in each case of the system, decreases, and/or a predicted number of containers that can be output in a unit of time, and/or a predicted accommodation capacity, in each case of the system, increases.

By adjusting the number of pasteurization zones to predicted system parameters, the heat treatment of the containers to be achieved is controlled as needed.

The control device can be designed to predict the transport speed, the number of containers entering in a unit of time, the output capacity, the number of containers that can be output in a unit of time, the accommodation capacity, and/or the system utilization in percent based on the/a transport speed, the/a number of containers entering in a unit of time, the/a number of containers that can be output in a unit of time, the/a length of the full part of an accumulation section, a number of (accumulated) containers in an accumulation section, the/an accommodation and/or output capacity, and/or the/a system utilization in percent, in each case of the system, and/or of one or more further systems upstream and/or downstream of the system in the past and/or present.

The reliability of the prediction of the system parameter of the system is improved by the dependence of the predicted system parameter of the system on one or more system parameters of the system and/or one or more further systems upstream and/or downstream of the system in the past and/or present.

The disclosure also provides a method for heating and/or cooling, in particular pasteurizing, closed containers filled with an in particular liquid filling material by means of a system described above. The method comprises transporting the containers in the transport direction, heating and/or cooling the containers according to the temperature profile along the transport direction, controlling the transport speed of the transport device and the temperature profile, wherein the transport speed changes over time between a stop and the nominal transport speed, predicting the system parameter, and controlling the temperature profile as a function of the predicted system parameter.

Such a method makes it possible to adjust the temperature profile to a predicted system parameter. The underlying object of reducing costs and energy consumption, as well as a negative impact of the heat treatment on the filling material due to possible overpasteurization, is thereby achieved.

According to the method, the transport speed can be controlled as a function of a number of containers in an infeed and/or outfeed of the system in the past and/or present, in particular the time curves thereof, and/or an available capacity for accommodating containers in the/an infeed and/or outfeed of the system in the past and/or present, in particular the time curves thereof.

By controlling the transport speed in this way, an accumulation or defect at the infeed and/or outfeed and thus a critical operational state and/or an operational state with increased energy consumption is avoided.

The method can comprise detecting containers in the region of the/an infeed and/or outfeed of the system, wherein the detection can comprise detecting an accumulation and/or capturing an image. In particular, the transport speed can thereby be controlled based on data of the detection.

By means of the detection in the region of the infeed and/or outfeed of the system, data about the system is collected in a simple manner, which data has a direct influence on the operational state of the system. It is thereby possible to control the transport speed as a function of the actual presence of containers in the region of the infeed and/or outfeed, in particular in the event of defects and/or accumulations in the infeed and/or outfeed. Critical operational states and/or operational states with increased energy consumption are thereby avoided in a simple manner.

The method can comprise predicting the system parameter based on a transport speed, a number of containers entering in a unit of time, a number of containers that can be output in a unit of time, the/a length of the full part of an accumulation section, a number of (accumulated) containers in an accumulation section, an accommodation and/or output capacity, and/or a system utilization in percent, in each case of the system, and/or of one or more further systems upstream and/or downstream of the system in the past and/or present.

The system parameter of the system can be reliably predicted due to the dependence of the predicted system parameter of the system on one or more system parameters of the system and/or one or more further systems upstream and/or downstream of the system in the past and/or present. An advantage of this type of prediction is that it requires no data other than known, inherent system parameters.

The method can comprise increasing a number of pasteurization zones if a) a predicted transport speed, a predicted number of containers entering in a unit of time, a predicted output capacity, and/or a predicted system utilization in percent, in each case of the system, increases, and/or b) a predicted number of containers that can be output in a unit of time, and/or a predicted accommodation capacity, in each case of the system, decreases. Alternatively or additionally, the method can comprise reducing a/the number of pasteurization zones if a) a predicted transport speed, a predicted number of containers entering in a unit of time, a predicted output capacity, and/or a predicted system utilization in percent, in each case of the system, decreases, and/or b) a predicted number of containers that can be output in a unit of time, and/or a predicted accommodation capacity, in each case of the system, increases.

By adjusting the number of pasteurization zones to predicted system parameters, the heat treatment of the containers to be achieved is controlled as needed.

The method can comprise predicting the transport speed, the number of containers entering in a unit of time, the output capacity, the number of containers that can be output in a unit of time, the accommodation capacity, and/or the system utilization in percent based on the/a transport speed, the/a number of containers entering in a unit of time, the/a number of containers that can be output in a unit of time, the/a length of the full part of an accumulation section, a number of (accumulated) containers in an accumulation section, the/an accommodation and/or output capacity, and/or the/a system utilization in percent, in each case of the system, and/or of one or more further systems upstream and/or downstream of the system in the past and/or present.

The reliability of the prediction of the system parameter of the system is improved by the dependence of the predicted system parameter of the system on one or more system parameters of the system and/or one or more further systems upstream and/or downstream of the system in the past and/or present.

### BRIEF DESCRIPTION OF THE FIGURES

The present disclosure will be explained in greater detail with reference to the following exemplary embodiments with reference to the figures, without limiting the disclosure to the specific embodiments shown. In the figures:

FIG. 1 schematically shows a system for heating and/or cooling, in particular pasteurizing, closed containers filled with an in particular liquid filling material in a side view and a corresponding temperature profile,

FIG. 2 is a block diagram during operation of the system of FIG. 1 for heating and/or cooling, in particular pasteurizing, closed containers filled with an in particular liquid filling material,

FIG. 3 schematically shows an example of a transport capacity of a pasteurizer over time, as well as a predicted heat output of the pasteurizer as a function of the output of an upstream filler.

### DETAILED DESCRIPTION

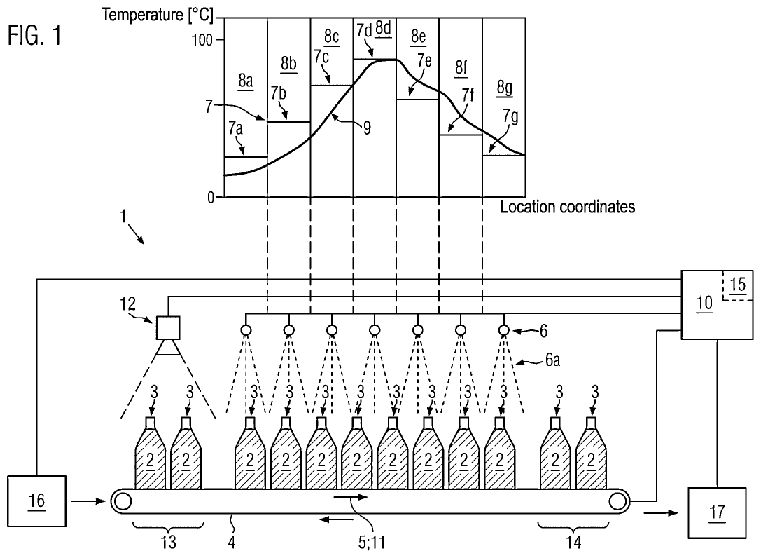

FIG. 1 illustrates the schematic structure of an embodiment of a system 1 for heating and/or cooling, in particular pasteurizing, closed containers filled with an in particular liquid filling material. In the system 1 shown, filled and closed containers are heated and/or cooled, in particular pasteurized, with an in particular liquid filling material.

Such a system 1 is used, for example, in a grouped plant in the food and/or consumer goods industry for the heat treatment of containers. Upstream, for example, a means for filling and closing the containers is provided. For example, a means for labeling the containers is arranged downstream.

FIG. 1 shows containers 3, for example bottles, filled with a filling material 2, for example juice or beer. The containers can also be cans or jars. The containers 3 are transported upright in a transport direction 5 on a transport device 4, for example a conveyor belt.

During transport, the containers 3 can be acted on from above with a liquid treatment medium 6a, for example water, from outfeed units of a device 6 for heating and/or cooling the containers 3.

The temperature of the liquid treatment medium 6a can, for example, be controlled zone by zone. A temperature profile 7 comprises controllable temperatures 7a-7g of temperature zones 8a-8g. For example, the device 6 comprises seven temperature zones 8a-8g. More or fewer, for example 5 to 12 temperature zones, can also be provided. The liquid treatment medium 6a of the specific temperature zone 8x has the specific temperature 7x, where x stands for a, b, c, d, e, f, or g. First temperature zones 8a-c that follow one another in the transport direction 5 are referred to as heating zones, for example. There can be, for example, three, four, or five or more temperature zones. The containers 3 are heated in these temperature zones 8a-c. The temperature zones 8a-8c are characterized, for example, by a stepwise increase in the corresponding temperatures 7a-7c. The temperature zone 8c is followed in the transport direction 5 by a temperature zone 8d, referred to as a pasteurization zone, the temperature 7d of which corresponds to a maximum temperature of the temperature profile or the temperature of which is so high that a pasteurization effect occurs. The pasteurization of the containers 3 takes place in the temperature zone 8d. The temperature 7d is the highest temperature below the temperatures 7a-7g. This means that both the temperatures 7a-7c and the temperatures 7e-7g are lower than the temperature 7d. The temperature zone 8d is followed in the transport direction 5 by the temperature zones 8e-8g, referred to as cooling zones, which follow one another in the transport direction 5. The containers can be cooled in these temperature zones 8e-8g. The temperature zones 8e-8g are characterized, for example, by a stepwise decrease in the corresponding temperatures 7e-7g.

If the containers 3 pass through the temperature profile 7 in the transport direction 5, a temperature of the filling material 2 changes according to a temperature curve 9. The temperature curve 9 was determined experimentally by measuring the core temperature in the center of a container 3. Starting at an initial temperature of the filling material 2 at the beginning of the temperature zone 8a, the temperature curve 9 increases in the temperature zones 8a-8d until the maximum temperature is reached in the temperature zone 8d in which the containers 3 are pasteurized. The temperature curve 9 decreases in the temperature zones 8e-8g.

The transport device 4 is controlled by a control device 10. This comprises that the control device 10 controls, for example, the transport speed 11 of the transport device 4 in a time change between a stop and a nominal transport speed. The control device may include instructions stored in memory of therein to carry out the operations described herein, including for example receiving signals from detection devices, which may be sensors, and sending actuation signals to actuators, such as heaters or coolers for controlling temperatures, and/or valves or motors for controlling transport speed.

The transport speed 11 can be controlled based on data from a detection device 12, for example an image capturing device, for example a camera. By way of example, the detection device 12 is arranged in the region of an infeed 13 of the system 1 and is provided for detecting the containers 3 in the region of the infeed 13 of the system 1. Alternatively or additionally, the/a detection device can be arranged in the region of an outfeed 14 of the system 1 for detecting the containers in the region of the outfeed 14 of the system 1. This last alternative is not discussed further below. The detection device 12 in the region of the infeed 13 of the system 1 can continuously collect data, for example images of the containers 3 in the region of the infeed 13. By known methods of image evaluation, a number of containers 3 in the infeed 13 can be determined from the data, for example.

The control device 10 can control the transport speed 11 of the transport device 4 as a function of the determined number of containers 3 in the infeed 13. This comprises, for example, that the control device 10 stops the transport device 4 if the number of containers 3 in the infeed 13 falls below a predefined minimum number, and controls the transport speed 11 to a nominal transport speed if the number of containers 3 in the infeed 13 exceeds a predefined maximum number. The minimum number is lower than the maximum number.

In addition, the temperature profile 7 can be controlled by the control device 10. In this case, controlling the temperature profile 7 comprises controlling the temperatures 7a-7g of the temperature zones 8a-8g. The temperature profile 7 is controlled, for example, as a function of a predicted system parameter 15, for example a transport speed of the system. The system parameter 15 is predicted by the control device 10, for example, based on a transport speed of an upstream system 16, for example a filling machine. Alternatively, the control device 10 can also predict the system parameter 15 based on a transport speed of a downstream system 17, for example a labeling machine. This last alternative is not discussed further below. The upstream system 16 sends information about its present transport speed to the control device. In this case, the control device can predict the transport speed of the system in the future, for example, based on this information and the time known to the control device 10 that the containers need in order to reach the system 1 from the upstream system 16. The control device 10 can then, for example, adjust or change the temperature profile 7 as a function of the predicted transport speed of the system 1. The control device 10 can, for example, increase the temperatures 7a-7g of individual temperature zones 8a-8g in such a way that a plurality of the temperature zones 8a-8g have the maximum temperature at which the pasteurization of the containers 3 takes place when the predicted transport speed of the system 1 increases compared to a previous value. On the other hand, the control device 10 can reduce the temperatures 7a-7g of individual temperature zones 8a-8g in such a way that fewer of the temperature zones 8a-8g have the maximum temperature at which the pasteurization of the containers 3 takes place when the predicted transport speed of the system 1 decreases compared to a preceding value. At any time, at least one of the temperature zones 8a-8g has a temperature required for pasteurizing the containers 3.

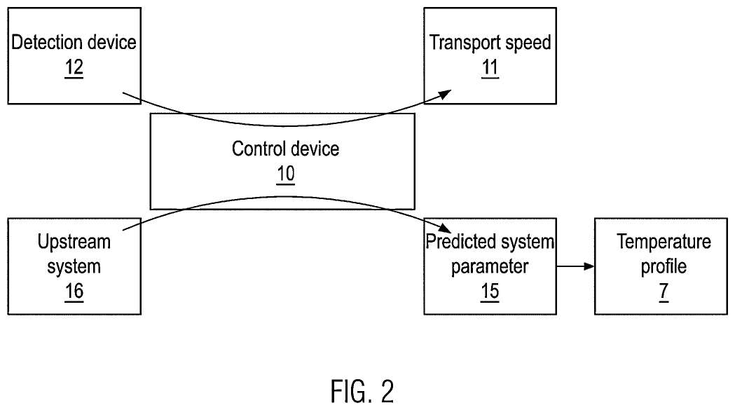

FIG. 2 shows the sequence of processes of the control device 10 during operation of the system 1 of FIG. 1 for heating and/or cooling, in particular pasteurizing, closed containers filled with an in particular liquid filling material in a block diagram.

On the one hand, the control device 10 receives data, for example images of the containers 3 in the region of the infeed 13, from the detection device 13, for example an image capturing device, for example a camera. By known methods of image evaluation, the control device 10 can determine a number of containers 3 in the infeed 13, for example from the data. The control device 10 can control the transport speed 11 of the transport device 4 as a function of the determined number of containers 3 in the infeed 13. This comprises that the control device 10 can stop the transport device 4 if the number of containers 3 in the infeed 13 falls below a predefined minimum number, and can control the transport speed 11 to a nominal transport speed if the number of containers 3 in the infeed 13 exceeds a predefined maximum number. The minimum number is lower than the maximum number.

On the other hand, the control device 10 receives information about a present transport speed of the upstream system 16. Based on this information and the time known to the control device 10 that the containers 3 need to reach the system 1 from the upstream system 16, the control device 10 can predict the system parameter 15, for example the transport speed of the system in the future. The control device 10 controls the temperature profile 7 in the future as a function of the predicted system parameter 15 of the system 1.

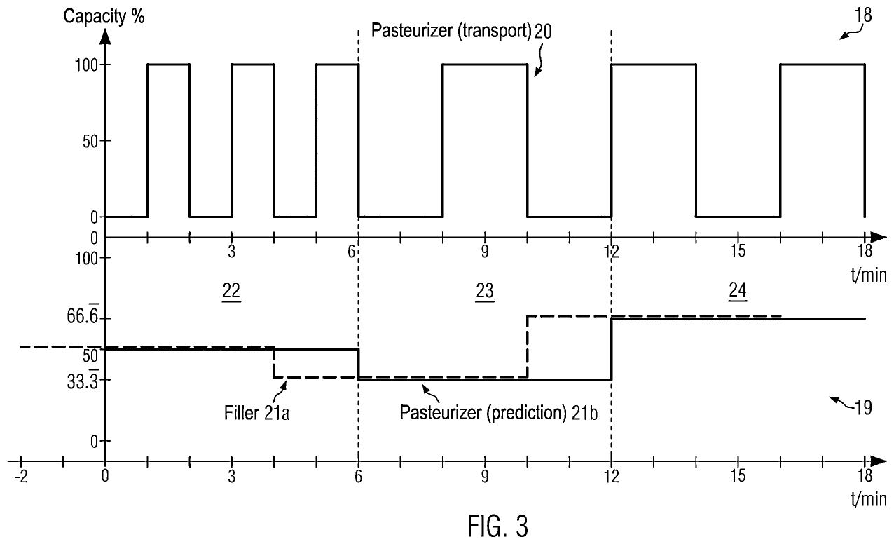

FIG. 3 schematically shows an example of a transport capacity 20 of a pasteurizer over time, as well as a predicted system utilization 21b of the pasteurizer as a function of the output of an upstream filler 21a.

The transport capacity 20 over time can correspond to the transport speed 11 over time of previous embodiments. The higher/lower the transport capacity 20 at a given time, the higher/lower is the corresponding transport speed 11. The pasteurizer can be an example of a system 1; the upstream filler can be an example of an upstream system 16 from previous embodiments. The predicted system utilization 21b of the pasteurizer can thereby be comprised in the predicted system parameter of previous embodiments. According to the predicted system utilization 21b of the pasteurizer/the predicted system parameter, the temperature profile 7 can be adjusted analogously to the embodiment of FIG. 1. In this example, the output of the upstream filler 21a can correspond to the transport speed of the filler. The higher/lower the output of the filler, the higher/lower is the corresponding transport speed.

In a lower part 19 of FIG. 3, the output 21a over time of the filler is shown by way of example. From t=−2 min to t=4 min, i.e., in this example a period of time of six minutes, the output of the filler is, for example, 50%. Subsequently, from t=4 min to t=10 min, the filler can run at one-third load at 33.3̅% output. From t=10 min to t=16 min, the output of the filler can be two thirds, i.e., 66.6̅%. In this example, the output over time of the filler 21a is two minutes before the predicted system utilization of the pasteurizer 21b. The time curve of the filler 21a can be used as a basis for predicting the system utilization of the pasteurizer 21b. In the example shown, the output over time of the filler 21a can be exactly as far ahead of the system utilization over time of the pasteurizer 21b as the containers 3 need in order to be transported from the filler to the pasteurizer, in this example two minutes. In the present case, the system utilization of the pasteurizer 21b can be predicted according to the output of the filler 21a. Consequently, the system utilization of the pasteurizer 21b is predicted in a first period of time 22 from t=0 min to t=6 min at 50%, in a second period of time 23 of t=6 min to t=12 min at 33.3̅%, and in a third period of time 24 of t=12 min to t=18 min at 66.6̅%. According to the predicted system utilization 21b of the pasteurizer/the predicted system parameter, in this example the temperature profile 7 can be adjusted analogously to the embodiment of FIG. 1.

In an upper part 20 of FIG. 3, the transport capacity over time of the pasteurizer 20 is shown to illustrate this example. In this example, the transport capacity of the pasteurizer 20, and thus the transport speed 11 over time, shows a change over time between a stop (0% capacity) and a nominal transport speed (100% capacity) over all periods of time 22, 23, and 24. The current transport capacity/transport speed of the pasteurizer can thereby be controlled as a function of a present number of containers in an infeed of the pasteurizer, analogously to the embodiment of FIG. 2. The transport capacity of the pasteurizer 20 in the example shown can correspond to the corresponding output of the upstream filler 21a via the control based on the present number of containers in the infeed of the pasteurizer with an offset of two minutes, in each case averaged over a period of time 22, 23, or 24. Continuous operation is thereby achieved. Alternating transport operation of the pasteurizer with more stop times corresponds to slow operation of the filler, and alternating transport operation of the pasteurizer with extended nominal load times corresponds to fast operation of the filler.

### LIST OF REFERENCE SIGNS

1 System
2 Filling material
3 Container
4 Transport device
5 Transport direction
6 Device
6a Treatment medium
7 Temperature profile
7a-7g Temperatures
8a-8g Temperature zones
9 Temperature curve
10 Control device
11 Transport speed
12 Detection device
13 Infeed
14 Outfeed
15 Predicted system parameter
16 Upstream system
17 Downstream system
18 Upper part
19 Lower part
20 Transport capacity of a pasteurizer
21a Output of an upstream filler
21b Predicted system utilization of a pasteurizer
22 First period of time
23 Second period of time
24 Third period of time

1. A system for heating and/or cooling closed containers filled with a filling material, comprising:
a transport device designed to transport the containers in a transport direction,
a device designed to heat and/or cool the containers according to a temperature profile along the transport direction, and
a control device designed to control a transport speed of the transport device and the temperature profile,
wherein the control device is designed to control the transport speed during a time change between a stop and a nominal transport speed,
wherein the control device is designed to control the temperature profile as a function of a predicted system parameter.

2. The system according to claim 1, wherein the system parameter comprises the transport speed, a number of containers entering in a unit of time, a number of containers that can be output in a unit of time, a length of the full part of an accumulation section, a number of (accumulated) containers in an accumulation section, an accommodation and/or output capacity, and/or a system utilization in percent, in each case of the system.

3. The system according to claim 1, wherein the control device is designed to control the transport speed as a function of:
a number of containers in an infeed and/or outfeed of the system in the past and/or present, and/or
an available capacity for accommodating containers in the/an infeed and/or outfeed of the system in the past and/or present.

4. The system according to claim 3, further comprising a detection device in the region of the/an infeed and/or outfeed of the system for detecting containers in the region of the infeed and/or outfeed of the system,
wherein the detection device comprises an accumulation switch and/or an image capturing device.

5. The system according to claim 1, wherein the control device is designed to predict the system parameter based on a transport speed, a number of containers entering in a unit of time, a number of containers that can be output in a unit of time, the/a length of the full part of an accumulation section, a number of (accumulated) containers in an accumulation section, an accommodation and/or output capacity, and/or a system utilization in percent, in each case of the system, and/or of one or more further systems upstream and/or downstream of the system in the past and/or present.

6. The system according to claim 1, wherein the device has a plurality of temperature zones along the transport direction of the containers,
wherein the temperature profile comprises controllable temperatures of the temperature zones and a control of the temperature profile comprises a control of the temperatures of the temperature zones,
wherein each temperature zone has a uniform temperature for itself.

7. The system according to claim 6, wherein in a plurality of temperature zones referred to as heating zones, which follow one another in the transport direction, the temperatures increase or remain constant from temperature zone to temperature zone, and
wherein the temperature corresponds to a maximum temperature in at least one or more temperature zones referred to as pasteurization zones following the heating zones in the transport direction, and
wherein the temperatures decrease or remain constant from temperature zone to temperature zone in a plurality of temperature zones referred to as cooling zones following the pasteurization zones in the transport direction.

8. The system according to claim 7, wherein the control device is designed to:
increase a number of pasteurization zones if
a) a predicted transport speed, a predicted number of containers entering in a unit of time, a predicted output capacity, and/or a predicted system utilization in percent, in each case of the system, increases, and/or
b) a predicted number of containers that can be output in a unit of time, and/or a predicted accommodation capacity, in each case of the system, decreases,
and/or
to reduce a/the number of pasteurization zones if
a) a predicted transport speed, a predicted number of containers entering in a unit of time, a predicted output capacity, and/or a predicted system utilization in percent, in each case of the system, decreases, and/or
b) a predicted number of containers that can be output in a unit of time, and/or a predicted accommodation capacity, in each case of the system, increases.

9. The system according to claim 8, wherein the control device is designed to predict the transport speed, the number of containers entering in a unit of time, the output capacity, the number of containers that can be output in a unit of time, the accommodation capacity, and/or the system utilization in percent based on the/a transport speed, the/a number of containers entering in a unit of time, the/a number of containers that can be output in a unit of time, the/a length of the full part of an accumulation section, a number of (accumulated) containers in an accumulation section, the/an accommodation and/or output capacity, and/or the/a system utilization in percent, in each case of the system, and/or of one or more further systems upstream and/or downstream of the system in the past and/or present.

10. A method for heating and/or cooling closed containers filled with a filling material by means of a system according to claim 1, comprising:
transporting the containers in the transport direction,
heating and/or cooling the containers according to the temperature profile along the transport direction,
controlling the transport speed of the transport device and the temperature profile, wherein the transport speed changes over time between a stop and the nominal transport speed,
predicting the system parameter, and
controlling the temperature profile as a function of the predicted system parameter.

11. The method according to claim 10, wherein the transport speed is controlled as a function of:
a number of containers in the/an infeed and/or outfeed of the system in the past and/or present, and/or
an available capacity for accommodating containers in the/an infeed and/or outfeed of the system in the past and/or present.

12. The method according to claim 10, comprising detecting containers in the region of the/an infeed and/or outfeed of the system,
wherein the detection comprises detecting an accumulation and/or capturing an image.

13. The method according to claim 10, comprising predicting the system parameter based on a transport speed, a number of containers entering in a unit of time, a number of containers that can be output in a unit of time, the/a length of the full part of an accumulation section, a number of (accumulated) containers in an accumulation section, an accommodation and/or output capacity, and/or a system utilization in percent, in each case of the system, and/or of one or more further systems upstream and/or downstream of the system in the past and/or present.

14. The method according to claim 10, further comprising:
increasing a number of pasteurization zones if
a) a predicted transport speed, a predicted number of containers entering in a unit of time, a predicted output capacity, and/or a predicted system utilization in percent, in each case of the system, increases, and/or
b) a predicted number of containers that can be output in a unit of time, and/or a predicted accommodation capacity, in each case of the system, decreases,
and/or
reducing a/the number of pasteurization zones if
a) a predicted transport speed, a predicted number of containers entering in a unit of time, a predicted output capacity, and/or a predicted system utilization in percent, in each case of the system, decreases, and/or
b) a predicted number of containers that can be output in a unit of time, and/or a predicted accommodation capacity, in each case of the system, increases.

15. The method according to claim 14, comprising predicting the transport speed, the number of containers entering in a unit of time, the output capacity, the number of containers that can be output in a unit of time, the accommodation capacity, and/or the system utilization in percent based on the/a transport speed, the/a number of containers entering in a unit of time, the/a number of containers that can be output in a unit of time, the/a length of the full part of an accumulation section, a number of (accumulated) containers in an accumulation section, the/an accommodation and/or output capacity, and/or the/a system utilization in percent, in each case of the system, and/or of one or more further systems upstream and/or downstream of the system in the past and/or present.

16. The system according to claim 1, wherein the heating and/or cooling includes pasteurizing closed containers filled with liquid filling material.
